(19) United States
(12) Patent Application Publication
(10) Pub. No.: US 2012/0033784 A1
(43) Pub. Date: Feb. 9, 2012

(54) X-RAY DETECTOR AND X-RAY COMPUTER TOMOGRAPHY SCANNER

(76) Inventors: Keiji Matsuda, Nasushiobara-shi (JP); Michito Nakayama, Utsunomiya-shi (JP); Shuya Nambu, Nasushiobara-shi (JP); Takashi Kanemaru, Nasushiobara-shi (JP); Atsushi Hashimoto, Yaita-shi (JP); Tomoe Sagoh, Utsunomiya-shi (JP)

(21) Appl. No.: 13/196,284

(22) Filed: Aug. 2, 2011

(30) Foreign Application Priority Data
Aug. 6, 2010 (JP) 2010-177905

(51) Int. Cl. A61B 6/03; G01T 1/17

(52) U.S. Cl. 378/19; 250/394

(57) ABSTRACT

According to one embodiment, an X-ray detector includes a plurality of detection packs and a plurality of heaters. Each of the detection packs includes a plurality of detection elements that detect X rays having passed through a subject and output a detection signal corresponding to the X rays and is arranged along a predetermined direction. Each of the heaters is provided corresponding to each of the detection packs and individually controls the temperature of each of the detection packs.

### CROSS-REFERENCE TO RELATED APPLICATIONS

This application is based upon and claims the benefit of priority from prior Japanese Patent Application No. 2010-177905, filed Aug. 6, 2010, the entire contents of which are incorporated herein by reference.

### FIELD

Embodiments described herein relate generally to an X-ray detector and an X-ray computer tomography scanner.

### BACKGROUND

As is generally known, an X-ray computer tomography (CT) scanner includes an X-ray tube and an X-ray detector and a tomogram of a subject is obtained by irradiating the subject with X rays generated by the X-ray tube, capturing X rays that have passed through the subject by the X-ray detector, and performing processing of a signal thereof.

In recent years, multi-slice type X-ray detectors are commercially available on the market. A multi-slice type X-ray detector includes a plurality of detection packs arranged on a substantial arc in a channel direction perpendicular to a body axis direction of the subject and one detection pack includes many detection elements arranged in a matrix form in the channel direction and a slice direction. An electric signal output from each detection pack is converted into a digital signal by a DAS (Data Acquisition System) and a tomogram is generated based on the signal.

As detection elements used in an X-ray detector, for example, a detection element composed of a fluorescent substance such as a scintillator that converts X rays into light and a photoelectric conversion element such as a photodiode that converts the light into a charge (electric signal) and a detection element composed of a semiconductor device that directly converts X rays into a charge are known.

Detection sensitivity of X rays of such a detection element fluctuates depending on the temperature. Thus, to obtain a tomogram with high precision, it is necessary to stabilize the temperature of each detection element of an X-ray detector to an optimal value during imaging.

In view of the above circumstances, the X-ray detector has a function to control the temperature of each detection element. This function has been realized by providing one large heater inside the X-ray detector and controlling the heater.

The heater is normally arranged below (opposite side of the X-ray incidence plane) each detection pack so that X rays entering the detection pack are not blocked. In this state, it is necessary to provide the DAS further below the heater or in a side direction of the heater, leading to a larger X-ray detector.

The heater controls the temperature of detection elements included in all detection packs at the same time and thus, an AC (alternating current) heater that can be driven at a relatively large capacity is used. X rays emitted from an X-ray tube pass through a human body and thus, the intensity thereof is limited to a minimum necessary level in view of a harmful effect on the health of the subject. Thus, the intensity of X rays entering the X-ray detector is very weak and the amount of charge of a signal output from each detection pack to the DAS is extremely small. Therefore, if an AC heater is used for temperature control of each detection pack, there is the possibility of deterioration of the SN ratio [signal to noise ratio] of a signal output from the detection pack due to noise thereof.

Such a problem can be solved by using a DC (direct current) driven heater. However, normally a DC heater is, compared with an AC heater, unfit for large-capacity heating and adequate temperature control capabilities cannot be obtained simply by replacing the heater of a conventionally structured X-ray detector with a DC heater.

When the temperature of all detection packs is controlled by one heater, there arises a problem of fluctuations in temperature of each detection pack depending on how heat is conducted from the heater to each detection pack or the like.

Therefore, a conventional structure to control the temperature of detection elements of an X-ray detector has many problems.

### BRIEF DESCRIPTION OF THE DRAWINGS

FIG. 1 is a block diagram showing an overall structure of an X-ray CT scanner according to a first embodiment;

FIG. 2 is a schematic diagram when an internal structure of the X-ray detector in the embodiment is viewed from a Z-axis direction;

FIG. 3 is a perspective view showing a state before a connector board is connected to a detection pack in the embodiment;

FIG. 4 is a perspective view when the detection pack and the connector board shown in FIG. 3 are viewed from an arrow C direction;

FIG. 5 is a perspective view of the state in which the detection pack and the connector board shown in FIG. 3 are mounted;

FIG. 6 is a block diagram showing electric circuits and the like of the X-ray detector in the embodiment;

FIG. 7 is a flow chart showing a control example of a heater by each heater controller in the embodiment;

FIG. 8 is a block diagram showing electric circuits and the like of the X-ray detector in a second embodiment;

FIG. 9 is a block diagram showing electric circuits and the like of the X-ray detector in a third embodiment;

FIG. 10 is a perspective view showing the state before the connector board is connected to the detection pack in a fourth embodiment;

FIG. 11 is a perspective view when the detection pack and the connector board shown in FIG. 10 are viewed from the arrow C direction;

FIG. 12 is a perspective view of the state in which the connector board and the detection pack shown in FIG. 10 are mounted;

FIG. 13 is a perspective view showing the state before the connector board is connected to the detection pack in a fifth embodiment;

FIG. 14 is a perspective view when the detection pack and the connector board shown in FIG. 13 are viewed from the arrow C direction; and

FIG. 15 is a perspective view of the state in which the connector board and the detection pack shown in FIG. 13 are mounted.

### DETAILED DESCRIPTION

In general, according to one embodiment, an X-ray detector includes a plurality of detection packs and a plurality of heaters. Each of the detection packs includes a plurality of detection elements that detect X rays having passed through a subject and output a detection signal corresponding to the X rays and is arranged along a predetermined direction. Each of the heaters is provided corresponding to each of the detection packs and individually controls the temperature of each of the detection packs.

Each embodiment will be described below with reference to the drawings. In the description that follows, the same reference numerals are attached to structural elements having substantially the same function and configuration and a duplicate description will be provided only when necessary.

### First Embodiment

First, the first embodiment will be described.

### [Overall Configuration of the X-Ray CT Scanner]

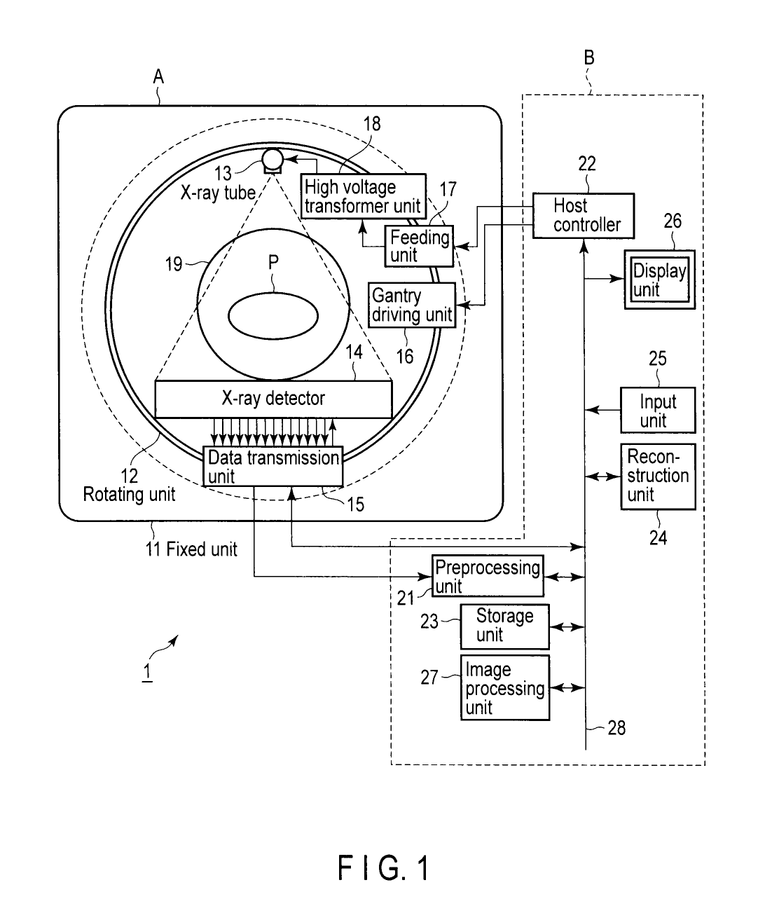

FIG. 1 is a block diagram showing an overall structure of an X-ray CT scanner 1 according to the first embodiment. As shown in FIG. 1, the X-ray CT scanner 1 includes a gantry unit A and a console unit B.

The gantry unit A acquires projection data (or original data) by irradiating a subject with X rays and detecting X rays that have passed through the subject. There are various type of imaging systems of an X-ray CT system such as a ROTATE/ROTATE type in which an X-ray tube and a two-dimensional detector system integrally rotate around a subject and a STATIONARY/ROTATE type in which many detection elements are arrayed in a ring shape and only the X-ray tube rotates around the subject, and an X-ray CT system of the ROTATE/ROTATE type, which is currently mainstream, is taken as an example.

As shown in FIG. 1, the gantry unit A includes a fixed unit 11, a rotating unit 12, an X-ray tube 13, an X-ray detector 14, a data transmission unit 15, a gantry driving unit 16, a feeding unit 17, and a high voltage transformer unit 18.

The central portion of the rotating unit 12 is open together with a cabinet and a subject P placed on a top board of a bed unit is inserted through an opening 19 thereof during imaging.

The X-ray tube 13 is a vacuum tube to generate X rays and is provided in the rotating unit 12. The X-ray detector 14 is used to detect X rays having passed through the subject P and is mounted on the rotating unit 12 in a direction opposite to the X-ray tube 13.

The gantry driving unit 16 rotates the rotating unit 12 around the subject P in the opening 19 at high speed. Accordingly, the X-ray tube 13 and the X-ray detector 14 integrally rotate around the center axis parallel to the body axis direction of the subject P inserted through the opening 19.

Operating power is supplied to the fixed unit 11 from an external power supply such as a commercial AC power supply. The operating power supplied to the fixed unit 11 is transmitted to the rotating unit 12 via the feeding unit 17. The feeding unit 17 supplies the operating power to each unit of the rotating unit 12. The high voltage transformer unit 18 includes a high voltage transformer, a filament heating converter, a rectifier, and a high voltage switch and transforms the operating power supplied from the feeding unit 17 into a high voltage, which is supplied to the X-ray tube 13.

Next, the console unit B will be described. The console unit B includes a preprocessing unit 21, a host controller 22, a storage unit 23, a reconstruction unit 24, an input unit 25, a display unit 26, an image processing unit 27, and data/control bus 28.

The preprocessing unit 21 receives original data from the X-ray detector 14 via the data transmission unit 15 and makes sensitivity corrections and X-ray intensity corrections of the original data. Original data on which preprocessing is performed by the preprocessing unit 21 is called “projection data”.

The host controller 22 exercises unified control of various kinds of processing such as imaging processing, data processing, and image processing.

The storage unit 23 stores image data such as collected original data, projection data, and CT image data.

The reconstruction unit 24 generates reconstruction image data for predetermined slices by performing reconstruction processing on projection data based on predetermined reconstruction parameters (the reconstruction region size, reconstruction matrix size, threshold to extract the region of interest and the like).

The input unit 25 includes a keyboard, various switches, and a mouse and an operator operates the input unit 25 to input various scan conditions such as the slice thickness and the number of slices.

The image processing unit 27 performs image processing for the display such as the window conversion and RGB processing on the reconstruction image data generated by the reconstruction unit 24 and outputs the data to the display unit 26. The image processing unit 27 also generates a tomogram of any section, projection image from any direction, and a so-called pseudo three-dimensional image such as a three-dimensional surface image and outputs such an image to the display unit 26. The output image data is displayed in the display unit 26 as an X-ray CT image.

The data/control bus 28 is a signal wire to transmit/receive various kinds of data, control signals, and address information by connecting each unit.

In the description below, the rotation axis of the rotating unit 12 is defined as the Z axis. In a rotating coordinate system around the Z axis, an axis connecting the focal point of the X-ray tube 13 and the center of the detection surface of the X-ray detector 14 and perpendicular to the Z axis is defined as the X axis and an axis perpendicular to the Z axis and the X axis is defined as the Y axis.

### [X-Ray Detector]

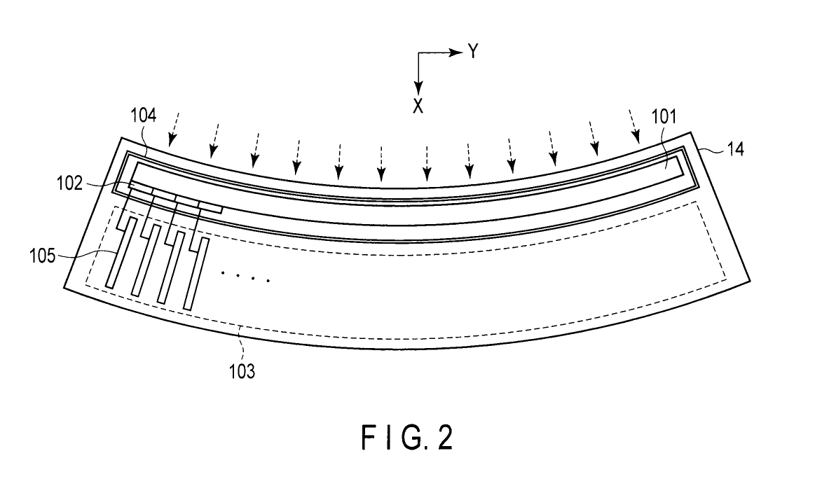

FIG. 2 is a schematic diagram showing an internal structure of the X-ray detector 14 when viewed from the Z-axis direction.

The X-ray detector 14 is formed in an arc shape around the X-ray tube 13 and includes a collimator unit 101, K (about 40, for example) detection packs 102 mounted on the collimator unit 101, a DAS unit 103 provided below each of the detection packs 102, and an insulation case 104 accommodating the collimator unit 101 and each of the detection packs 102.

The collimator unit 101 has a known structure in which many collimator plates are mounted on a support member in an arc shape around the X-ray tube 13.

The detection packs 102 are one-dimensionally arrayed in the Y axis direction and mounted on the support member of the collimator unit 101. On the side of the X-ray irradiation surface of the detection packs 102, many detection elements are arrayed in a matrix shape of M×N (about 64×24, for example) regarding the slice direction (Z axis direction) and the channel direction substantially perpendicular thereto (substantial Y axis direction).

The DAS unit 103 includes K DAS boards 105 electrically connected to each of the detection packs 102. Each of the DAS boards 105 is electrically connected to one detection pack 102 and performs amplification processing and A/D conversion processing on an analog signal (detection signal) output from the connected detection pack 102 when X rays are detected to generate a predetermined digital signal and outputs the signal to the data transmission unit 15. In FIG. 2, only a portion of the K detection packs 102 and DAS boards 105 is illustrated.

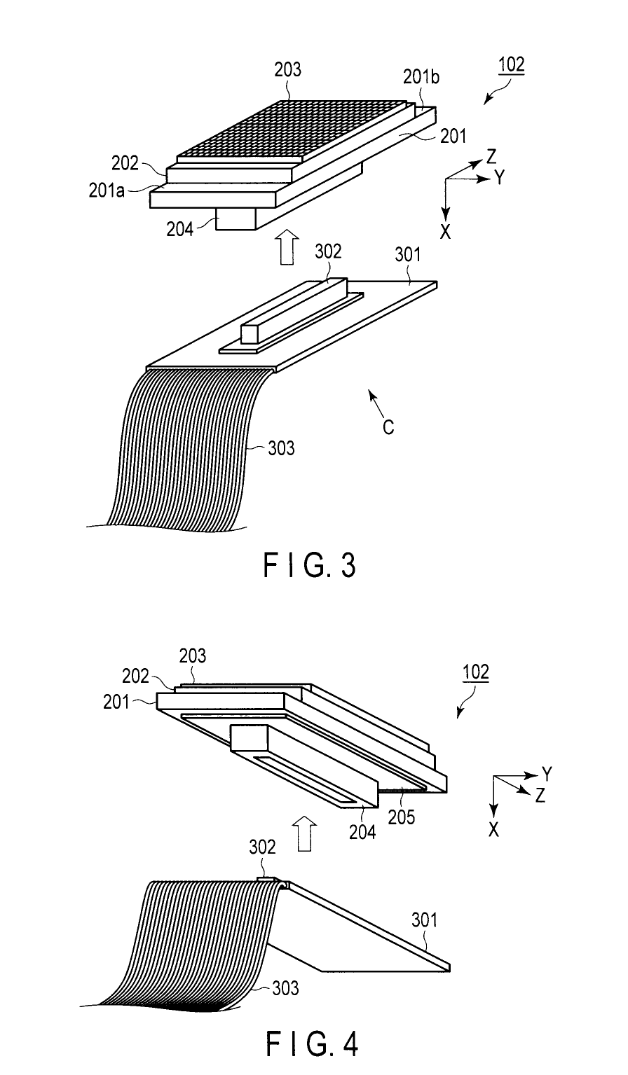

The insulation case 104 is formed of, for example, a material with high insulation properties such as a resin material and ceramic. An insulation port of a flexible cable 303 (see FIG. 3) to connect each of the detection packs 102 and each of the DAS boards 105 is provided on a surface opposite to the X-ray incidence plane of the insulation case 104 or the side face thereof.

In a conventional X-ray detector, for example, one AC heater in a flat long shape is provided below (DAS unit side) each detection pack and the temperature of the insulation case is controlled by the AC heater.

### [Detection Pack]

Next, the detection pack 102 will be described in detail with reference to FIGS. 3 and 4.

FIG. 3 is a perspective view showing a state before a connector board 301 for connection to the DAS board 105 is connected to the detection pack 102 and FIG. 4 is a perspective view when the detection pack 102 and the connector board 301 shown in FIG. 3 are viewed from an arrow C direction.

The detection pack 102 in the present embodiment is configured by mounting a flat photodiode board 202 on the top surface of a base board 201 and placing a scintillator block 203 on the top surface of the board 202. Further, as shown in FIG. 4, a pack-side connector 204 (first connector) and a DC driven heater 205 are mounted on the undersurface of the base board 201.

The base board 201 has a flat shape prolonged in the Z axis direction. The width of the photodiode board 202 in the Z axis direction is a little smaller than that of the base board 201 and the width of the scintillator block 203 in the Z axis direction is a little smaller than that of the photodiode board 202. The base board 201, the photodiode board 202, and the scintillator block 203 all have substantially the same width in the Y direction. Therefore, if the base board 201, the photodiode board 202, and the scintillator block 203 are mounted by aligning the center of each in the ZY plane, as shown in FIG. 3, the side wall in the Y axis direction becomes flush with each other and margin surfaces 201a, 201b are formed on the top surface of the base board 201. When mounted on the collimator unit 101, the margin surfaces 201a, 201b are brought into close contact with the support member of the collimator unit 101 and fixed by a predetermined method such as screwing. At this point, the side walls in the Y axis direction of the detection packs 102 are brought into close contact so that no gap arises on the X-ray detection surface between the adjacent detection packs 102.

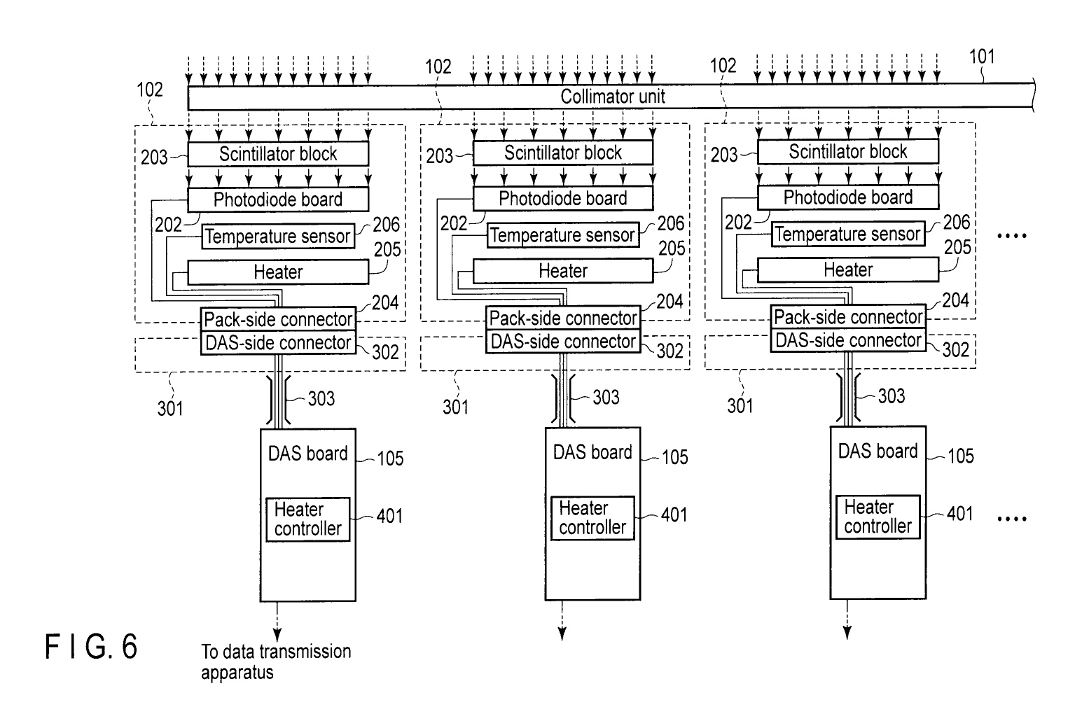

The base board 201 contains a temperature sensor 206 (see FIG. 6) to detect the temperature of a detection element group.

The scintillator block 203 is configured by arraying scintillators converting X rays into visible light in an array form (M×N). The photodiode board 202 has photodiodes as photoelectric conversion elements formed in an array form (M×N) so as to correspond to the scintillators. Thus, one detection element is configured by one scintillator of a scintillator block and one photodiode corresponding thereto.

The heater 205 has a substantially flat shape with an opening in a joint portion of the pack-side connector 204 and the base board 201 and operates by receiving the supply of DC from the side of the DAS board 105 via the pack-side connector 204. The heater 205 is a small heater with widths in the Z axis direction and the Y axis direction not exceeding those in the Z axis direction and the Y axis direction of the base board 201 respectively.

The pack-side connector 204 has an output terminal group to output an analog signal from each detection element, a connection terminal for the heater 205, and an output terminal for the temperature sensor 206. A DAS-side connector 302 (second connector) provided on the flat connector board 301 to connect each of the terminals of the pack-side connector 204 is mounted on the pack-side connector 204. The wide flexible cable 303 (communication cable) extending from the DAS board 105 is connected to the connector board 301. The flexible cable 303 is a bundle of a signal wire (M×N) to transmit an analog signal from each detection element to the DAS board 105, a power supply line to supply power from the DAS board 105 to the heater 205, and a signal wire to transmit output from the temperature sensor 206 to the DAS board 105. As the pack-side connector 204, the DAS-side connector 302, and the flexible cable 303, for example, standard products having as many terminals and signal wires as the number obtained by adding the number of the power supply line for the heater 205 and the signal wire for the temperature sensor to the number of signal wires (M×N) to transmit the analog signal or more may be used.

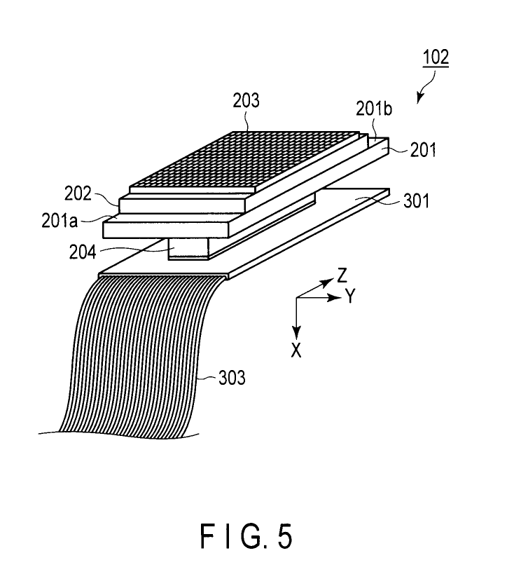

A perspective view of the state in which the connector board 301 is mounted on the detection pack 102 is shown in FIG. 5. If the DAS-side connector 302 is mounted on the pack-side connector 204 in this manner, each detection element, the heater 205, and the temperature sensor 206 are each connected to the DAS board 105 electrically.

All of the K detection packs 102 have the configuration described by using FIGS. 3 to 5.

### [Electric Circuit and Heater Control of the X-Ray Detector]

FIG. 6 is a block diagram showing electric circuits and the like of the X-ray detector 14.

When X rays emitted from the X-ray tube 13 enter the X-ray detector 14, unnecessary scattered X rays are eliminated by the collimator unit 101. Each scintillator of the scintillator block 203 emits light after receiving X rays after scattered X rays being eliminated and each photodiode provided on the photodiode board 202 outputs an electric signal (analog signal) through photoelectric conversion after receiving visible light from the scintillator. Thus, the analog signal output from each detection element and a signal indicating the temperature detected by the temperature sensor 206 are sent to the DAS board 105 connected to each of the detection packs 102 via the pack-side connector 204, the DAS-side connector 302, the connector board 301, and the flexible cable 303.

Each of the DAS board 105 in the present embodiment is provided with a heater controller 401. Each of the heater controllers 401 is realized by a control circuit of each of the DAS boards 105 and controls the heater 205 of the detection pack 102 connected to each of the DAS boards 105 by fluctuating the current value supplied to the heater 205.

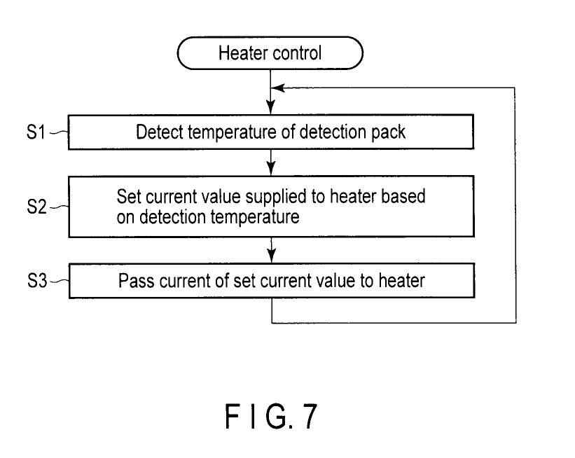

FIG. 7 shows a control example of the heater 205 by each of the heater controllers 401. The processing shown here is performed independently by the heater controller 401 of each of the DAS boards 105.

In this control example, first the heater controller 401 detects a temperature T of the detection pack 102 connected to the heater controller 401 based on a signal output from the temperature sensor 206 (step S1).

Subsequently, the heater controller 401 sets a current value I to be supplied to the heater 205 of the detection pack 102 connected to the heater controller 401 based on a detection temperature T (step S2). While the current value I is set by aiming for a temperature at which the detection pack 102 can obtain satisfactory X-ray detection characteristics as a target value in this processing, various methods can be adopted as a concrete setting method. For example, a table associating the current value I to be set with the detection temperature T may be stored in a memory in the DAS board 105 so that the current value I is set by referring to the table. Also, the current value I may be set by substituting the detection temperature T into a predetermined formula. Alternatively, if the detection temperature T falls below the target value, the current value I may be set to a value higher than the current value currently supplied to the heater 205 by a predetermined value and if the detection temperature T exceeds the target value, the current value I may be set to a value lower than the current value currently supplied to the heater 205 by a predetermined value or to zero. When the current value I is set by using a table or formula as described above, the concrete value of the current value I for the detection temperature T may be determined in consideration of theoretically or experimentally derived relationships between the detection temperature T and the current value I.

If the current value I is set in this manner, the heater controller 401 supplies the current of the current value I to the heater 205 of the detection pack 102 connected to the heater controller 401 (step S3). The processing in steps S1 to S3 is repeated in a predetermined period while the X-ray CT scanner 1 is in a standby state of imaging.

As described above, the small heater 205 is mounted on each of the detection packs 102 in the present embodiment and the temperature of each of the detection packs 102 is controlled by the heater 205. If such a configuration is adopted, compared with a case when a large heater is provided below each detection pack in the past, the space inside the X-ray detector 14 can be saved.

Moreover, by providing the heater 205 for each of the detection packs 102 in this manner, adequate temperature control capabilities can be obtained even if a DC heater is adopted as the heater 205. Therefore, the temperature of each of the detection packs 102 can be controlled without using a large-output AC-driven heater and noise from the heater 205 or a power supply line to the heater 205 will not be entrapped into an analog signal output from each of the detection packs 102.

As a result of prevention of noise from the heater 205 or a power supply line to the heater 205 from being entrapped, the signal wire to transmit the analog signal from each of the detection packs 102 to each of the DAS boards 105 and the power supply line of the heater 205 can be bundled so that the DAS board 105 can be made a power supply source of the heater 205. Therefore, there is no need to separate the line connecting each of the detection packs 102 and each of the DAS boards 105 into a plurality of lines or to provide a control circuit dedicated to the heater 205 inside the X-ray detector 14. This also contributes to space saving inside the X-ray detector 14.

Because the temperature of each of the detection packs 102 is individually controlled by each of the heaters 205, fluctuations in temperature of each of the detection packs 102 can be corrected more easily.

### Second Embodiment

Next, the second embodiment will be described.

The present embodiment is different from the first embodiment in that the heater 205 of each of the detection packs 102 is controlled by one heater controller in a unified manner, instead of controlling the heater 205 of each of the detection packs 102 individually by providing the heater controller 401 for each of the DAS boards 105. The reference numerals are attached to the same locations as those in the first embodiment and a description thereof will not be repeated.

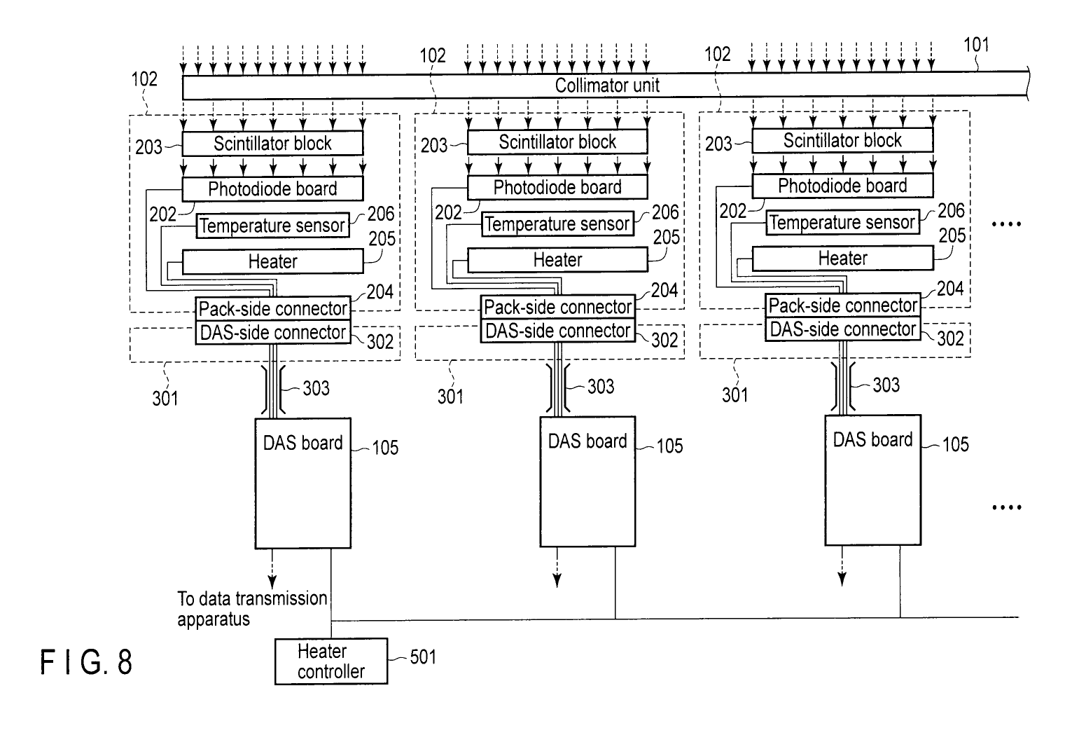

FIG. 8 is a block diagram showing electric circuits and the like of the X-ray detector 14 in the present embodiment.

As shown in FIG. 8, the X-ray detector 14 is provided with a heater controller 501 electrically connected to each of the DAS boards 105. The configurations of the detection pack 102 and the connector board 301 are the same as those in the first embodiment.

With such a configuration, the heater controller 501 controls each of the heaters 205 basically in the same flow as that of the control example shown in FIG. 7. First, the heater controller 501 detects temperatures T₁ to T_{K} of each of the detection packs 102 based on a signal output from each of the temperature sensors 206 (step S1).

Subsequently, the heater controller 501 sets current values I₁ to I_{K} to be supplied to each of the heaters 205 based on the detection temperatures T₁ to T_{K} (step S2). The current value Iₓ (1≧x≧K) is the current value to be supplied to the heater 205 provided in the detection pack 102 in which the temperature Tₓ (1≧x≧K) is detected. In the present embodiment, after heating by each of the heaters 205 is started, the current values I₁ to I_{K} are set so that the detection temperatures T₁ to T_{K} become substantially uniform, even before each of the detection temperatures T₁ to T_{K} is stabilized at a target value. Various methods can be adopted as a concrete setting method. For example, as described in the first embodiment, each of the current values I₁ to I_{K} is set so that each of the detection temperatures T₁ to T_{K} approaches a target value and then, these current values I₁ to I_{K} are corrected so that fluctuations of the detection temperatures T₁ to T_{K} are rectified. In these corrections, for example, the current value supplied to the heater 205 of the detection pack 102 whose detection temperature is relatively low is slightly increased and the current value supplied to the heater 205 of the detection pack 102 whose detection temperature is relatively high is slightly decreased.

After the current values I₁ to I_{K} are set in this manner, the heater controller 501 supplies currents of the current values I₁ to I_{K} to the heaters 205 of the corresponding detection packs 102 (step S3). The processing in steps S1 to S3 is repeated in a predetermined period while the X-ray CT scanner 1 is active.

As described above, the heater 205 of each of the detection packs 102 is controlled by the one heater controller 501 in the present embodiment. Even with such a configuration, like in the first embodiment, the space inside the X-ray detector 14 can be saved. Moreover, a DC-driven heater can be adopted as each of the heaters 205 and thus, noise from the heater 205 or a power supply line to the heater 205 will not be entrapped into an analog signal output from each of the detection packs 102.

If fluctuations in detection temperature of each of the temperature sensors 206 arise, each of the heaters 205 can be driven to correct the fluctuations and thus, the temperature of each of the detection packs 102 can be maintained uniform immediately after temperature control of each of the detection packs 102 is started even before each of the detection temperatures T₁ to T_{K} is stabilized at a target value. Therefore, even when, for example, a sufficient temperature control time cannot be secured before imaging due to an urgent diagnosis, local deterioration of a CT image will not occur.

### Third Embodiment

Next, the third embodiment will be described.

The present embodiment is different from the second embodiment in that the temperature sensor 206 is provided in a portion of the detection packs 102, instead of providing the temperature sensor 206 in all the detection packs 102. The reference numerals are attached to the same locations as those in the first and second embodiments and a description thereof will not be repeated.

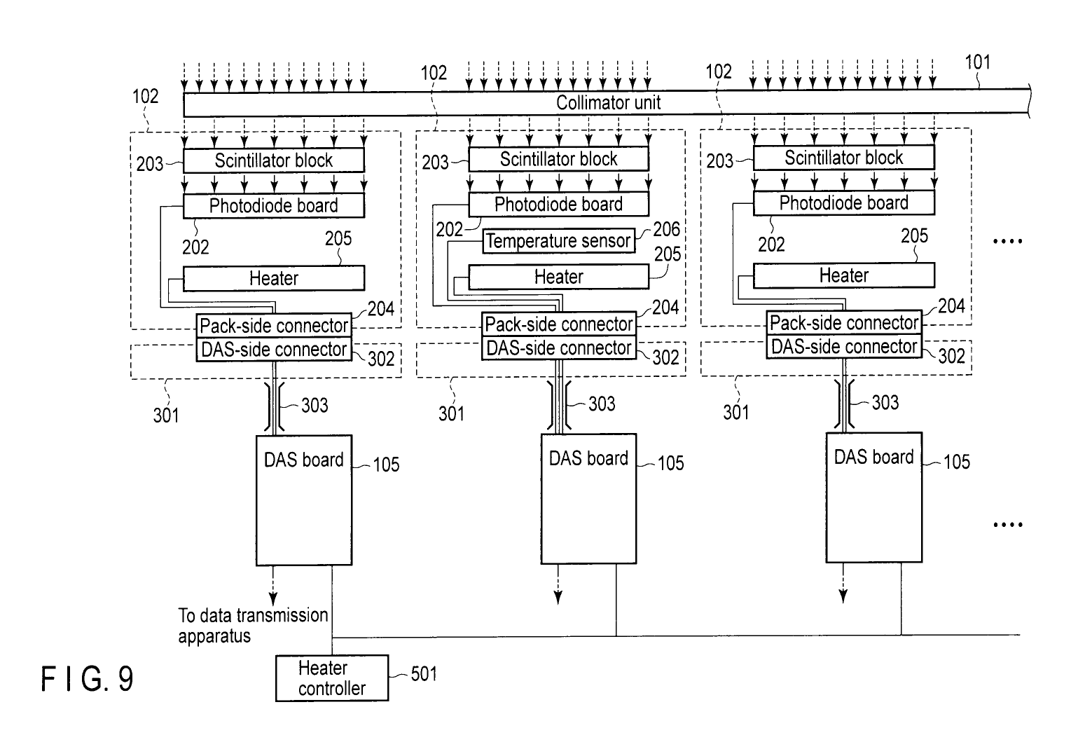

FIG. 9 is a block diagram showing electric circuits and the like of the X-ray detector 14 in the present embodiment.

As shown in FIG. 9, of the three detection packs 102 arranged consecutively, the detection pack 102 positioned in the center is provided with the temperature sensor 206 and the two detection packs 102 adjacent to the detection pack 102 are not provided with the temperature sensor 206. That is, if, of the K detection packs 102, the detection pack 102 arranged at one end of the X-ray detector 14 is defined as the first and the detection pack 102 arranged at the other end as the K-th, the temperature sensor 206 is provided in the second, fifth, eighth, eleventh, . . . detection packs 102. The detection pack 102 provided with the temperature sensor 206 and the two adjacent detection packs 102 are defined as a group below and it is assumed that L such groups are present in the X-ray detector 14.

With the configuration described above, the heater controller 501 controls each of the heaters 205 basically in the same flow as that of the control example shown in FIG. 7. First, the heater controller 501 detects temperatures T_{G1} to T_{GL} of the detection packs 102 arranged in the center of each group based on a signal output from each of the temperature sensors 206 (step S1).

Subsequently, the heater controller 501 sets current values I_{G1} to I_{GL} to be supplied to the heater 205 in each group based on the detection temperatures T_{G1} to T_{GL} (step S2). The current value I_{GX} (1≧x≧L) is the current value to be supplied to the heater 205 in the group to which the detection pack 102 in which the temperature Tₓ (1≧x≧L) is detected belongs. Various methods can be adopted as the setting method of the current values I_{G1} to I_{GL} in the processing. For example, as described in the first embodiment, each of the current values I_{G1} to I_{GL} may be set so that each of the detection temperatures T_{G1} to T_{GL} approaches a target value and further, as described in the second embodiment, each of the current values I_{G1} to I_{GL} may be corrected so that fluctuations of the detection temperatures T_{G1} to T_{GL} are rectified.

After the current values I_{G1} to I_{GL} are set in this manner, the heater controller 501 supplies currents of the current values I_{G1} to I_{GL} to the heaters 205 in the corresponding groups (step S3). The processing in steps S1 to S3 is repeated in a predetermined period while the X-ray CT scanner 1 is active.

As described above, the temperature sensor 206 is provided in a portion of the detection packs 102 in the present embodiment and detection temperatures of these temperature sensors 206 are used to control the heater 205 of each of the detection packs 102. With the configuration described above, there is no need to provide the temperature sensor 206 in all the detection packs 102 and thus, the control configuration is simplified and also manufacturing costs of the X-ray detector 14 can be reduced.

Even with the configuration in the present embodiment, like in the first embodiment, the space inside the X-ray detector 14 can be saved. Moreover, a DC-driven heater can be adopted as each of the heaters 205 and thus, noise from the heater 205 or a power supply line to the heater 205 will not be entrapped into an analog signal output from each of the detection packs 102.

### Fourth Embodiment

Next, the fourth embodiment will be described.

The present embodiment is different from each of the above embodiments in that the heater is provided on the connector board 301, instead of in the detection pack 102. The same reference numerals are attached to the same structural elements as those in each of the above embodiments and a description thereof will not be repeated.

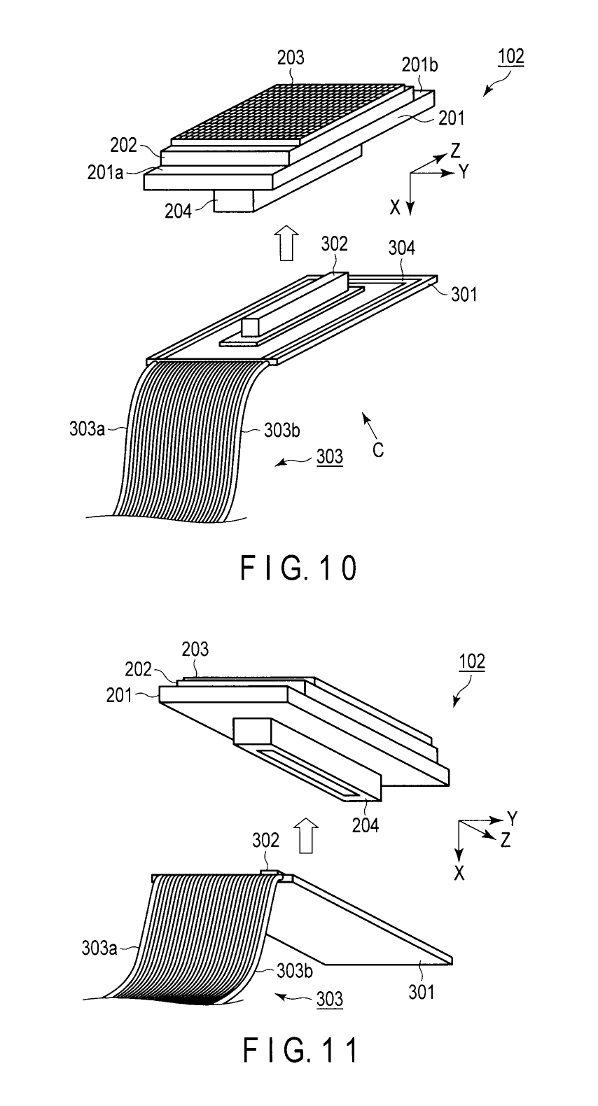

FIG. 10 is a perspective view showing a state before the connector board 301 for connection to the DAS board 105 is connected to the detection pack 102 and FIG. 11 is a perspective view when the detection pack 102 and the connector board 301 shown in FIG. 10 are viewed from the arrow C direction.

As described above, the detection pack 102 is not provided with a heater (see FIG. 11). On the other hand, the connector board 301 is provided with a DC-driven heater 304 bent along an outer edge thereof. Power supply lines 303a, 303b to supply a current to the heater 304 are provided at both ends of the flexible cable 303. Then, the power supply line 303a is connected to one end of the heater 304 and the power supply line 303b is connected to other end thereof.

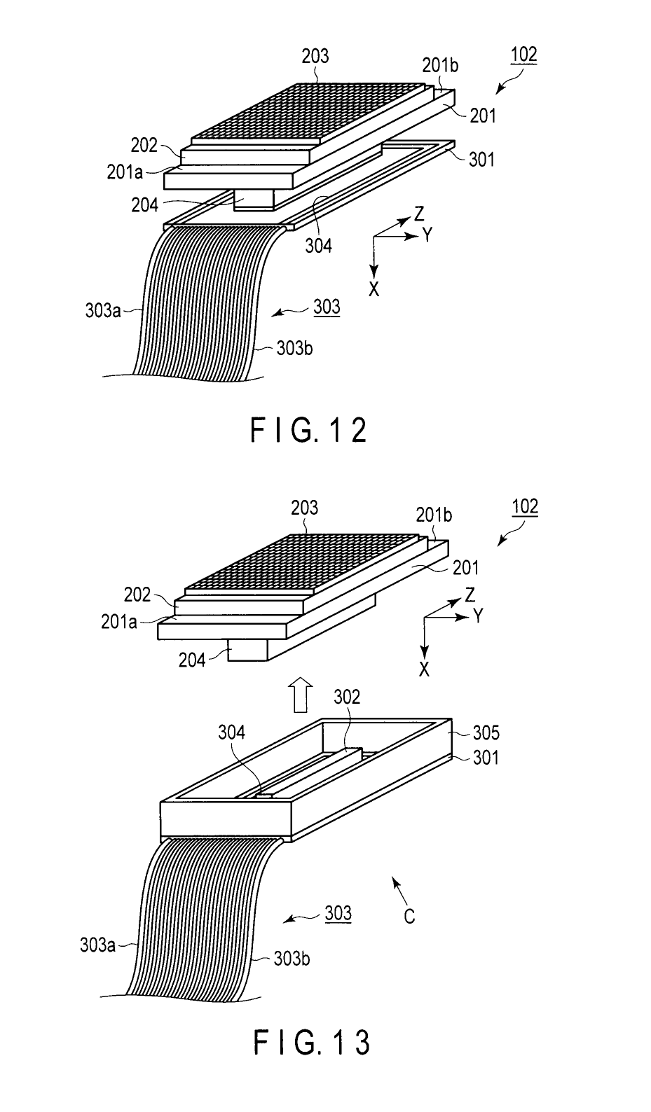

A perspective view of the state in which the connector board 301 configured as described above is mounted on the detection pack 102 is shown in FIG. 12. If the connector board 301 is mounted on the detection pack 102, as shown in FIG. 12, the heater 304 faces the detection pack 102 with a gap formed therebetween. In this state, the heater controller 401 in the first embodiment or the heater controller 501 in the second/third embodiment performs the processing shown in FIG. 7 and when a current is supplied to each of the heaters 304, heat generated by each of the heaters 304 is transmitted through an air space in the gap to heat each of the detection packs 102.

All of the K detection packs 102 have the configuration described by using FIGS. 10 to 12.

In the present embodiment, the heater 304 provided in each of the detection packs 102 may be controlled, like in the first embodiment, by the separate heater controllers 401 or, like in the second embodiment, by the one heater controller 501. Also in the present embodiment, the temperature sensor 206 may be provided, like in the first/second embodiments, in each of the detection packs 102 or, like in the third embodiment, in a portion of the detection packs 102.

As described above, the heater 304 is provided on the connector board 301 mounted on the detection pack 102 in the present embodiment, instead of providing the heater in the detection pack 102. With the configuration described above, there is no need to provide heater terminals in the pack-side connector 204 and the DAS-side connector 302. Further, even if an error such as breaking of wire occurs in the heater, the error can be handled by replacing the connector board 301 that is cheaper than a detection pack so that maintenance costs of the X-ray CT scanner 1 can be held down.

### Fifth Embodiment

Next, the fifth embodiment will be described.

The present embodiment is different from the fourth embodiment in that a cover member covering the gap between the detection pack 102 and the connector board 301 is provided on the connector board 301. The same reference numerals are attached to the same structural elements as those in each of the above embodiments and a description thereof will not be repeated.

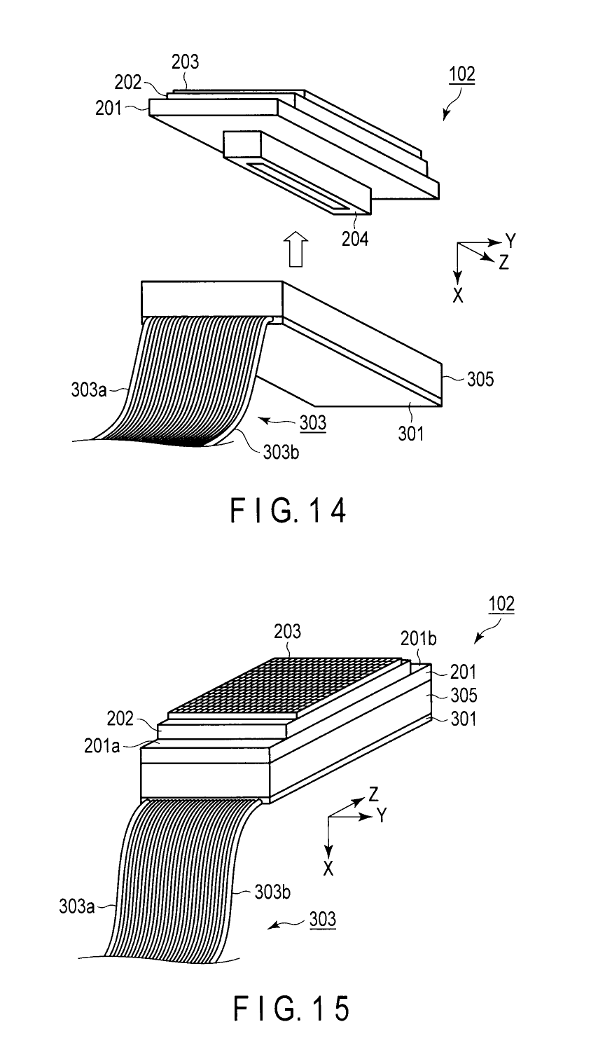

FIG. 13 is a perspective view showing a state before the connector board 301 for connection to the DAS board 105 is connected to the detection pack 102 and FIG. 14 is a perspective view when the detection pack 102 and the connector board 301 shown in FIG. 13 are viewed from the arrow C direction.

A cover member 305 in a rectangular frame shape is provided on the surface on the side of the connector board 301 on which the DAS-side connector 302 along a circumference thereof. The cover member 305 is formed of, for example, a material with high insulation properties such as a resin material and ceramic and the height thereof substantially matches the width of the gap formed between the detection pack 102 and the connector board 301 when the connector board 301 is mounted on the detection pack 102.

A perspective view of the state in which the connector board 301 configured as described above is mounted on the detection pack 102 is shown in FIG. 15. If the connector board 301 is mounted on the detection pack 102, as shown in FIG. 15, the gap formed between the detection pack 102 and the connector board 301 is covered with the cover member 305. In this state, the heater controller 401 in the first embodiment or the heater controller 501 in the second/third embodiment performs the processing shown in FIG. 7 and when a current is supplied to each of the heaters 304, heat generated by each of the heaters 304 is transmitted through an air space in the gap to heat each of the detection packs 102. Since the gap is covered with the cover member 305, heat generated by the heater 304 efficiently warms the detection pack 102 without being lost to the surroundings.

All of the K detection packs 102 have the configuration described by using FIGS. 13 to 15.

In the present embodiment, the heater 304 provided in each of the detection packs 102 may be controlled, like in the first embodiment, by the separate heater controllers 401 or, like in the second embodiment, by the one heater controller 501. Also in the present embodiment, the temperature sensor 206 may be provided, like in the first/second embodiments, in each of the detection packs 102 or, like in the third embodiment, in a portion of the detection packs 102.

In the present embodiment, as described above, the cover member 305 to cover a gap formed between each of the detection packs 102 and each of the connector boards 301 is provided. With such a configuration, the detection pack 102 is efficiently warmed by the heater 304 and thus, the temperature of each of the detection packs 102 is swiftly controlled and also power consumption of the X-ray CT scanner 1 is reduced.

According to the configurations disclosed in the first to fifth embodiments, suitable effects such as being able to save space inside the X-ray detector 14, reducing entrapment of noise from the heater 205 or a power supply line to the heater 205 into an analog signal output from each of the detection packs 102, and being able to correct fluctuations in temperature of each of the detection packs 102 are achieved.

### (Modifications)

The configurations disclosed in each of the above embodiments can be embodied by appropriately modifying each structural element in the stage of implementation. Concrete modifications are, for example, as follows:

(1) In each of the above embodiments, a case when the DAS unit 103 includes as many the DAS boards 105 as the number of the detection packs 102 is illustrated. However, one DAS board 105 may be connected to a plurality of the detection packs 102 to reduce the number of the DAS boards 105. In such a case, each of the DAS boards 105 may process analog signals output from the plurality of the detection packs 102 connected to the DAS board 105. Further, if, like in the first embodiment, the heater controller 401 is realized by a control circuit of the DAS board 105, the heater controller 401 of each of the DAS boards 105 may drive each of the heaters 205 of the plurality of the detection packs 102 connected to the DAS board 105.
(2) In each of the above embodiments, a case when a detection element of the detection pack 102 is composed of scintillators and photodiodes is illustrated. However, the detection element may be configured by other methods such as using a semiconductor device that directly converts X rays into a charge.
(3) In the third embodiment, a case when one group is defined by the three detection packs 102 and the detection pack 102 arranged in the center of each group is provided with the temperature sensor 206 is illustrated. However, similar heater control may be exercised by defining the two, four or more detection packs 102 as a group or the temperature sensor 206 in each group may be provided in the detection pack 102 arranged at an end, instead of the detection pack 102 arranged in the center thereof.

Further, only one temperature sensor 206 may be provided for all the detection packs 102 so that the heater 205 of each of the detection packs 102 is controlled based on the detection temperature by the temperature sensor 206. Even in such a case, the temperature of each of the detection packs 102 can unchangingly be controlled by a DC-driven heater so that a noise reduction effect similar to that in each of the above embodiments can be gained.

(4) In the fourth embodiment, a case when the heater 304 bent in a “ [symbol] ” shape is provided along an outer edge of the connector board 301 is illustrated. However, the heater 304 provided on the connector board 301 may be a heater in a flat shape as shown in the first embodiment or a heater meandering along an outer edge of the connector board 301.
(5) In the fifth embodiment, a case when the cover member 305 is provided on the connector board 301 is illustrated. However, the cover member 305 may be provided on the detection pack 102, instead of the connector board 301.
(6) In each of the above embodiments, the heaters 205, 304 are assumed to be DC driven. However, an AC-driven heater may be adopted as the heaters 205, 304. Even in such a case, it is unchangingly possible to save space in the X-ray detector 14 and the temperature of each of the detection packs 102 can be maintained uniform. Moreover, there is no need to use a large AC heater as in the past and thus, entrapment of noise into an analog signal output from each of the detection packs 102 can also be reduced.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray detector, comprising: a plurality of detection packs including a plurality of detection elements that detect X rays having passed through a subject to output a detection signal corresponding to the X rays and arrayed along a predetermined direction; and a plurality of heaters provided corresponding to each of the detection packs to control a temperature of each of the detection packs individually.

2. The X-ray detector according to claim 1, wherein each of the heaters operates by receiving a supply of a direct current.

3. The X-ray detector according to claim 1, further comprising: a heater controller that controls each of the heaters.

4. The X-ray detector according to claim 3, further comprising: a temperature sensor that detects a temperature of each of the detection packs, wherein the heater controller controls each of the heaters based on a detection temperature of the temperature sensor.

5. The X-ray detector according to claim 3, further comprising: a DAS unit that converts the detection signal output from each of the detection packs into a digital signal, wherein the heater controller is realized by a control circuit constituting the DAS unit.

6. The X-ray detector according to claim 4, wherein the temperature sensor is provided for each of the detection packs and the heater controller controls each of the heaters so that the detection temperature of each of the temperature sensors becomes uniform.

7. The X-ray detector according to claim 4, wherein a group is formed for each of a predetermined number of the detection packs, one temperature sensor is provided for each group, and the heater controller controls each of the heaters based on the detection temperature of the temperature sensor corresponding to the group to which the detection pack whose temperature is controlled by each heater belongs.

8. The X-ray detector according to claim 1, wherein each of the detection packs is provided with a first connector having an output terminal of the detection signal output from each of the detection elements, further comprising: a plurality of connector boards on which a second connector attached at and detached from the first connector of each of the detection packs; and a DAS unit connected to each of the connector boards by a predetermined communication cable to convert the detection signal output from the first connector of each of the detection packs via the connector board and the communication cable into a digital signal, wherein each of the heaters is provided on each of the connector boards.

9. The X-ray detector according to claim 8, further comprising: a cover member provided on each of the detection packs or each of the connector boards to cover a gap formed between the detection pack where the first connector is provided and the connector board where the second connector is provided when the first connector and the second connector are connected.

10. The X-ray detector according to claim 8, wherein the communication cable is a bundle of at least a signal wire to transmit the detection signal output from the first connector of the detection pack to the DAS unit and a power supply line to supply power to the heater provided for the detection pack.

11. An X-ray CT scanner, comprising: an X-ray tube that generates X rays; a plurality of detection packs including a plurality of detection elements that detect X rays having passed through a subject to output a detection signal corresponding to the X rays and arrayed along a predetermined direction; and a plurality of heaters provided corresponding to each of the detection packs to control a temperature of each of the detection packs individually.

12. The X-ray CT scanner according to claim 11, wherein each of the heaters operates by receiving a supply of a direct current.

13. The X-ray CT scanner according to claim 11, further comprising: a heater controller that controls each of the heaters.

14. The X-ray CT scanner according to claim 13, further comprising: a temperature sensor that detects a temperature of each of the detection packs, wherein the heater controller controls each of the heaters based on a detection temperature of the temperature sensor.

15. The X-ray CT scanner according to claim 13, further comprising: a DAS unit that converts the detection signal output from each of the detection packs into a digital signal, wherein the heater controller is realized by a control circuit constituting the DAS unit.

16. The X-ray CT scanner according to claim 14, wherein the temperature sensor is provided for each of the detection packs and the heater controller controls each of the heaters so that the detection temperature of each of the temperature sensors becomes uniform.

17. The X-ray CT scanner according to claim 14, wherein a group is formed for each of a predetermined number of the detection packs, one temperature sensor is provided for each group, and the heater controller controls each of the heaters based on the detection temperature of the temperature sensor corresponding to the group to which the detection pack whose temperature is controlled by each heater belongs.

18. The X-ray CT scanner according to claim 11, wherein each of the detection packs is provided with a first connector having an output terminal of the detection signal output from each of the detection elements, further comprising: a plurality of connector boards on which a second connector attached at and detached from the first connector of each of the detection packs; and a DAS unit connected to each of the connector boards by a predetermined communication cable to convert the detection signal output from the first connector of each of the detection packs via the connector board and the communication cable into a digital signal, wherein each of the heaters is provided on each of the connector boards.

19. The X-ray CT scanner according to claim 18, further comprising: a cover member provided on each of the detection packs or each of the connector boards to cover a gap formed between the detection pack where the first connector is provided and the connector board where the second connector is provided when the first connector and the second connector are connected.

20. The X-ray CT scanner according to claim 18, wherein the communication cable is a bundle of at least a signal wire to transmit the detection signal output from the first connector of the detection pack to the DAS unit and a power supply line to supply power to the heater provided for the detection pack.
